(19) United States
(12) Patent Application Publication
(10) Pub. No.: US 2003/0158465 A1
(43) Pub. Date: Aug. 21, 2003

(54) Method and system for facilitating compliance with a dietary restriction

(76) Inventor: Doreen L. Galli, Smyrna, GA (US)

Correspondence Address: Gregory S. Bernabeo, Esq., Synnestvedt & Lechner LLP, 2600 Aramark Tower, 1101 Market Street, Philadelphia, PA, 19107-2950, US

(21) Appl. No.: 10/076,381

(22) Filed: Feb. 15, 2002

(51) Int. Cl. A61B 5/00

(52) U.S. Cl. 600/300

(57) ABSTRACT

A method, system and computer program product for facilitating compliance with a dietary restriction. Preparation criteria for the dietary restriction are compared to preparation notes for a food item. A dietary solution is provided that is compatible with the dietary restriction, e.g. to approve or disallow a food item, or to provide a preparation plan for preparing the food item to make it compatible with the dietary restriction. An identified ingredient or dietary restriction may be expanded be searching for related ingredients or characteristics. A customized menu may be prepared. The method may be implemented via a distributed computing network, and databases may be established to store data relating to ingredients, related ingredients, food preparation procedures, etc. in relation to dietary restrictions and/or food items.

### FIELD OF THE INVENTION

The present invention relates generally to a method and computer system for facilitating compliance with a dietary restriction.

### BACKGROUND OF THE INVENTION

Many people have diets that are subject to dietary restrictions relating to food allergies, health conditions, religious beliefs or personal preference. Such dietary restrictions may encourage or discourage consumption of food having various characteristics. For example, such characteristics may include ingredients (milk, dairy, etc.), handling procedures (use of utensils in according to kosher practices), or classifications (low-salt, high-fiber, kosher, vegetarian, etc.), to be sought or avoided. The need for adherence to a dietary restriction is particularly severe for those with food allergies because a single violation of the dietary restriction may result in extreme discomfort or death.

Adherence to dietary restrictions is often difficult. When preparing one's own meals, it may be easy to avoid some readily-identifiable ingredients (such as red meat), but it is more difficult to avoid others, particularly when the ingredient in one's recipe is a prepared food (such as chocolate) that may contain unknown ingredients or be prepared according to unknown procedures.

While many pre-prepackaged foods are provided with nutritional information and/or a list of ingredients, adherence to dietary restrictions when using prepackaged foods requires considerable sophistication. This is particularly true when an allergen may be commonly known by one name, but may appear in a list of ingredients under a variety of alternative names, or names of related ingredients, or may be implied by a description in an ingredient list (e.g. —“natural flavors”). For example, if one is allergic to milk, one must be able to identify alternative names and/or related ingredients that may appear on an ingredients list, such as caseinates, lactose, sodium lactate, cheese, yogurt, whey, milk, ice cream, buttermilk, etc. As used herein, “related ingredients” include those ingredients that have a common characteristic, or are related to one another, e.g. as derivatives, components, or as containing such derivatives or components. These related ingredients be wrongly identified as acceptable for consumption by someone who has an allergy to milk. For example, a list of related ingredients for a milk allergy may include all those items to which someone having a food allergy is likely allergic, e.g. caseinates, lactose, sodium lactate, cheese, yogurt, whey, milk, ice cream, buttermilk, etc.

The problem of identifying foods that do not comply with a dietary restriction is particularly severe in restaurants, hotels, hospitals, on airplanes, or elsewhere where a food item's ingredients and preparation procedures (e.g. the oil used for frying) are typically unknown to its consumer. Additionally, handling procedures are typically unknown to the consumer in such settings. Handling procedures can contaminate a food item or otherwise make it incompatible with a dietary restriction. For example, use of a cooking utensil may make a food item incompatible with a dietary restriction even if the cooking utensil is used properly or in accordance with usual food preparation standards. For example, a generally accepted food preparation practice may not be sufficient to prevent cross-contamination of foods to which one is allergic, or comply with desired food preparation procedures, such as preparation in a kosher manner.

Interrogating restaurant personnel as to ingredients and preparation and handling (collectively “preparation”) procedures may not provide satisfactory results because such personnel themselves may not be aware of preliminary food processing steps taken before the food reaches the restaurant. This is particularly common in fast-food or franchise chain restaurants. For example, many potatoes are oiled in a pre-processing step at a manufacturing center before they are distributed to the individual restaurants. Accordingly, restaurant personnel may be unaware of the type of oil applied to the potato, or may be unaware of any such oiling altogether.

Applicant has identified a need for a system for facilitating compliance with dietary restrictions, including restrictions relating to foods, ingredients, related ingredients, and food preparation procedures, particularly for foods prepared by others.

### SUMMARY OF THE INVENTION

The present invention provides a system, method and computer program product for facilitating compliance with dietary restrictions by comparing preparation criteria for the dietary restriction to preparation notes for a food item, and providing a dietary solution indicating whether or how the food item may be consumed in compliance with the dietary restriction. For example, an ingredient, pre-packaged food, or prepared food item may be approved or disallowed for consumption in accordance with a particular diner's dietary restriction. A database storing dietary restrictions in association with dietary characteristics and a database storing food items in association with dietary characteristics may be maintained for this purpose. An identified ingredient or dietary restriction may be expanded be searching for related ingredients or characteristics. A database of ingredients and related ingredients may be maintained for this purpose. Optionally, a customized menu is prepared to show foods as approved or disallowed, or including only approved items that may be a subset of a larger menu or list. When a food is disallowed or otherwise found to be incompatible with a dietary restriction, a preparation plan may be prepared that will make the food item compatible with the dietary restriction, e.g. by providing instructions to exclude an objectionable ingredient, or to ensure handling of the food in an approved manner compatible with the dietary restriction. A database of optional ingredients, substitutions, alternative processing instructions, etc. may be maintained for this purpose.

In a preferred embodiment, the method is implemented via a distributed computing network, such as the Internet, and is provided with a World Wide Web-based or other graphical user interface. In this manner, when preparation notes identifying ingredients, processing methods, handling procedures, etc. for food items of individual restaurants or restaurant chains is provided, an individual may obtain a dietary solution, or identify food items compatible with the individual's dietary restriction, before traveling to the restaurant. This is particularly useful for planning purposes, especially when traveling to a foreign city, etc.

More specifically, the present invention provides a method for facilitating compliance with a dietary restriction. The method includes the step of identifying preparation criteria associated with the dietary restriction and identifying preparation notes for the food item. The method further includes the step of comparing the preparation criteria to the preparation notes to determine compatibility, and providing a dietary plan for consumption of the food item that complies with the dietary restriction.

A system and computer program product are also provided.

### DESCRIPTION OF THE DRAWINGS

FIG. 1 is a flow diagram of an overview of a method for facilitating compliance with a dietary restriction in accordance with the present invention.

FIG. 2 is a flow diagram of an exemplary embodiment of the present invention in which related ingredients are identified.

FIG. 3 is a flow diagram of another exemplary embodiment of the present invention in which a preparation plan and customized menu are provided.

FIG. 4 is a block diagram of an information processing system in accordance with the present invention.

### DETAILED DESCRIPTION

The present invention provides a system and method for facilitating compliance with dietary restrictions by comparing preparation criteria for the dietary restriction to preparation notes for a food item and providing a dietary solution indicating whether or how the food item may be consumed in compliance with the dietary restriction. As used herein, “preparation criteria” may include any characteristics to be sought or avoided for a corresponding dietary restriction and “preparation notes” may include any characteristics of a corresponding food item. Whether a food item is “compatible” may be determined by any suitable standard. For example, standards may be used to require complete exclusion of an undesired ingredient, or to tolerate a limited or defined amount of an undesired ingredient, etc. Optionally, the standard may be specified by the user, the dietary restriction, the system, etc.

In accordance with the present invention, an individual having a dietary restriction due to allergy, health, religious, or other reasons may determine whether a certain food item may be consumed, or how preparation of a food item must be modified to be consumed, in accordance with the dietary restriction. For example, a person having a particular food allergy may identify the allergy and determine whether a specified prepackaged frozen dinner may be safely eaten. Alternatively, the person may determine whether there are any food items that should be avoided, or can be safely eaten, at a specified restaurant.

In some embodiments, related ingredients are identified and checked against a food item so that an accurate assessment of compatibility may be made. In this manner, a person allergic to dairy products is given a dietary solution that considers whether foods contain milk-related ingredients to which the person is likely allergic, such as caseinates, lactose, sodium lactate, cheese, yogurt, whey, milk, ice cream, buttermilk, etc. Accordingly, an expanded list of objectionable ingredients may be identified and considered to provide a highly accurate dietary plan.

Optionally, when it is determined that a food item is not typically prepared in a manner compatible with a dietary restriction (due to its ingredients or preparation steps), a preparation plan is provided that will make the food item compatible with the dietary restriction. For example, the preparation plan may provide instructions to exclude certain objectionable ingredients, or to handle the food in a certain manner.

It should be noted that the present invention is applicable to any characteristic, including ingredients, processing steps, preparation procedures, or other classifications, such as high-fat, high-sodium, non-kosher, non-vegetarian, etc. Any suitable characteristics may be considered, as will be apparent to those skilled in the art.

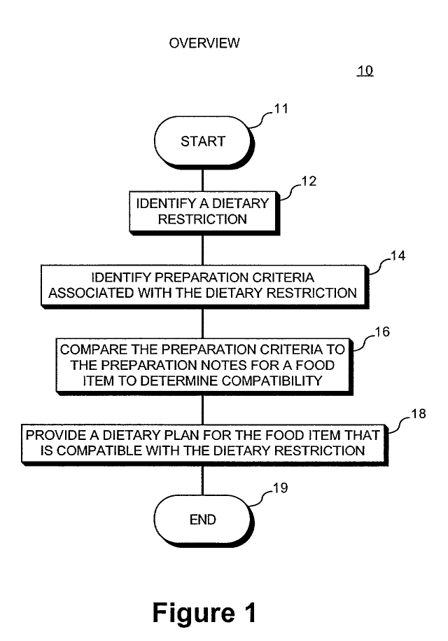

FIG. 1 is a flow diagram 10 of an overview of a method for facilitating compliance with a dietary restriction in accordance with the present invention. For example, the method of FIG. 1 may be used by an individual to determine whether he may safely eat a “Chocolate Delight” dessert at a “Big Burger” restaurant if he is allergic to dairy products. As shown in FIG. 1, the method starts with identification of a dietary restriction, as shown at steps 11 and 12. The dietary restriction may identify any characteristic, such as ingredients or preparation procedures, to be sought or avoided. Exemplary dietary restrictions include “dairy allergy,” “no milk,” “no dairy products,” “low fat only,” “high in fiber,” “kosher only”, “vegetarian only,”etc.

An individual may identify a dietary restriction directly or provide information from which a dietary restriction may be identified. For example, an individual may communicate a dietary restriction to an operator of the inventive system, or present a swipe card storing data identifying the dietary restriction. Advantageously, the individual may operate a home computer to transmit via a communications network a signal identifying the individual that may be used to identify a dietary restriction for the individual by referencing a database of user profiles, etc. Alternatively, the individual may operate a similar computer in a kiosk at a restaurant, etc.

Preparation criteria for the dietary restriction are then identified, as shown at step 14. The preparation criteria identifies characteristics for the dietary restriction that are used to determine whether a food item is compatible with a dietary restriction. For example, this step may be achieved by referencing a database of dietary restrictions to identify an ingredient to be avoided (e.g. milk), processing note (e.g. do not cross-contaminate with peanuts), etc.

The preparation criteria are then compared to preparation notes for a food item, as shown at step 16. The food item may be specified by a user, considered because it appears on a menu at a restaurant specified by a user, is a pre-packaged food the user wishes to purchase, etc. For example, the preparation notes may identify characteristics of the food item such as ingredients used in the food item (e.g. “milk”), preparation procedures for the food item (e.g. “fried with peanut oil,” “kosher ingredients,” “prepared in a kosher manner”) and/or classifications of the food item (e.g. “low fat,” “not high fat”) etc. These characteristics are used to determine whether the food item is compatible with the preparation criteria, and therefore the dietary restriction.

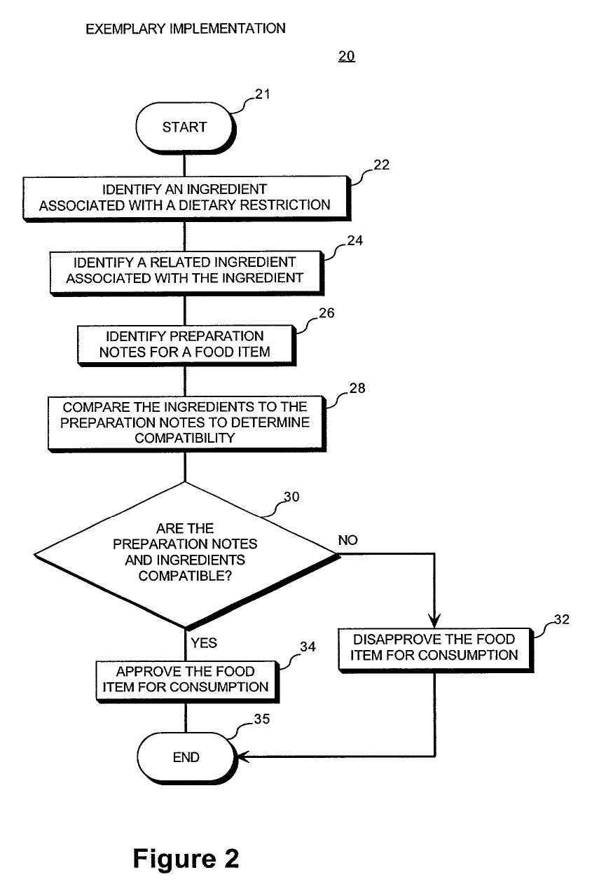

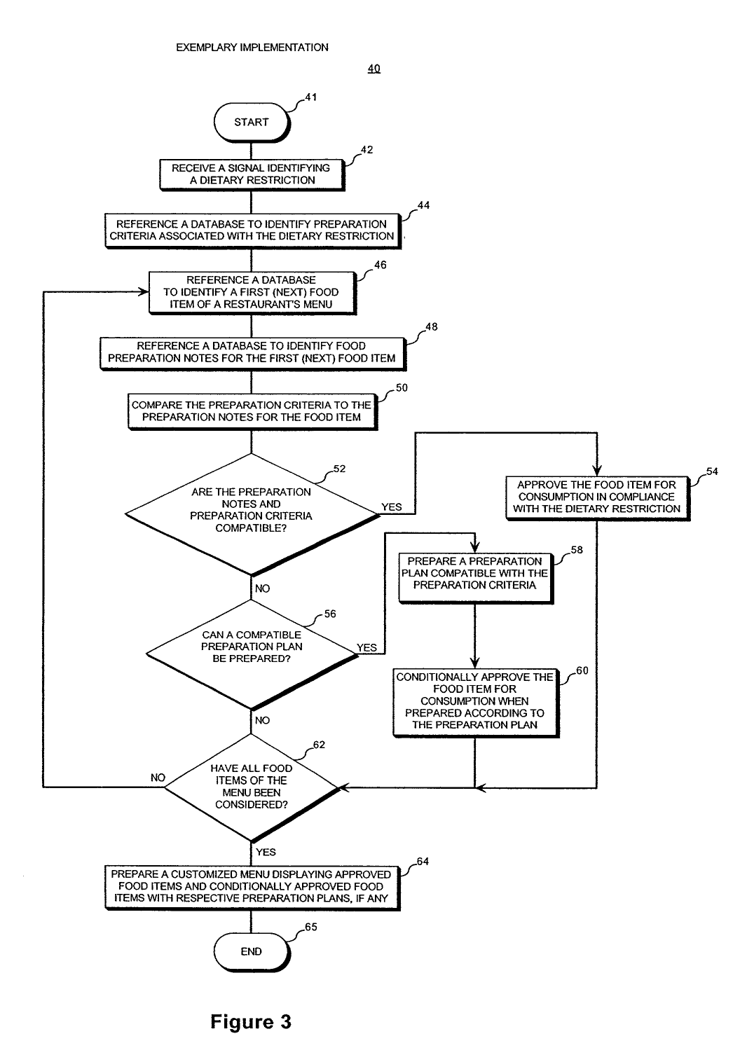

The method ends with providing of a dietary plan for the food item, as shown at steps 18 and 19. As used herein, a “dietary plan” is any suitable information indicating whether, how, when, etc. a food item should be consumed, prepared, etc. In certain embodiments, the dietary plan approves or disallows the food item for consumption in compliance with the dietary restriction depending upon whether the preparation criteria and preparation notes are compatible. Indications of approval and/or disallowance may be made in any suitable manner as will be apparent to those skilled in the art. For example, the dietary plan may provide a textual recommendation, a menu of foods compatible with the dietary restriction, a menu in which selected food items are identified as incompatible with the dietary restriction, indicia of approval, such as a checkmark or smiling face, or indicia of disallowance, such as an X, or a frowning face. In other embodiments, the dietary plan conditionally approves the food item for consumption and provides a preparation plan for the food item that makes the food item compatible with the dietary restriction. The dietary plan may be communicated in any suitable manner and have any suitable form, as will be apparent to those skilled in the art. Examples are discussed in below with reference to FIGS. 2 and 3.

Accordingly, if an individual identifies a dietary restriction indicating an allergy to dairy products, and milk is identified as an objectionable ingredient in the preparation criteria, and milk is identified as an ingredient in the preparation notes for the Big Burger's Chocolate Delight dessert, then a dietary plan is provided that disallows the dessert for the individual, indicating that the dessert should not be eaten, or provides conditional approval of the dessert and a preparation plan including instructions to exclude the milk from the dessert, indicating that the individual may eat the dessert if it is made without milk.

FIG. 2 is a flow diagram 20 of an exemplary embodiment of the present invention in which related ingredients are identified. The identification of related ingredients expands upon a known list of characteristics, e.g. objectionable ingredients, to provide a sophisticated, more-comprehensive analysis of food items in view of dietary restrictions. The identification of related ingredients is often helpful when an allergen is commonly known by one name, but may appear in a list of ingredients under a variety of names, or may be implied by a related ingredient. For example, if an individual identifies as an allergy to an ingredient, e.g. “milk,” as a dietary restriction, identification of related ingredients, such caseinates, lactose, sodium lactate, cheese, yogurt, whey, milk, ice cream, buttermilk, etc., may be identified and compared to the preparation notes of a food item. In this manner, a highly accurate dietary plan may be prepared.

As shown in FIG. 2, the method starts with identifying an ingredient (as a type of preparation criteria) associated with a dietary restriction, as shown at steps 21 and 22. This step may involve identifying a dietary restriction and referencing a database to identify the ingredient. Alternatively, an ingredient may be directly identified by a user, e.g. by selecting an ingredient from a menu of a graphical user-interface, typing the name of an ingredient to provide input to a system, etc.

A related ingredient associated with the identified ingredient is then identified, as shown at step 24. For example, this may be achieved by referencing a database storing related ingredients in association with ingredients. For example, if “milk” is specified by a user (or by a system after referencing a database of dietary restrictions) as an objectionable ingredient, and the food item contains the related ingredient “lactose”, a simple comparison would not recognize that the food item cannot be consumed in compliance with the dietary restriction. However, if the milk-related ingredients caseinates, lactose, sodium lactate, cheese, yogurt, whey, milk, ice cream, buttermilk, etc. are identified in this step, these related ingredients are treated as additional preparation criteria and a food item that contains “lactose” will properly be recognized as a food item that cannot be consumed in compliance with the dietary restriction.

Preparation notes for a food item are then identified, as shown at step 26. In this example, an individual specifies a food item a database of preparation notes is referenced to identify the preparation notes. For example, the individual may identify a “SuperChunk” candy bar or a restaurant's “Mexican Platter” meal and the corresponding preparation notes identifying the characteristics of the food item, including its ingredients and preparation notes, etc. will be referenced. For example, a restaurant chain may analyze its food and food preparation procedures and store appropriate data in a preparation database so that food items on its menu may be evaluated in accordance with the present invention. It should be noted that the present innovation may be implemented without revealing the ingredients, etc. to the user. This is advantageous when it is desired to maintain recipes, preparation procedures, etc. in secrecy.

The ingredients, including any related ingredients, are then compared to the preparation notes to determine compatibility, as shown at step 28. For example, consider that the dietary restriction is “no dairy products”, the objectionable ingredient is “milk,” and a related ingredient is “lactose.” In this example, if a food item's preparation notes identify “milk” or “lactose” as an ingredient, then the preparation criteria and preparation notes would be determined to be incompatible with the dietary restriction.

If the ingredients and preparation notes are compatible, then the food item is approved for consumption in compliance with the dietary restriction, as shown at steps 30 and 34. For example, the item may be approved for consumption by storing data in a database indicating approval, by displaying on a video display screen an approval message or indicia of approval such as a smiling face, and the method ends, as shown at step 35.

If the ingredients and preparation notes are not compatible, then the food item is disallowed for consumption by storing data in a database indicating disallowance by displaying on a video display screen a disallowance message or indicia of disallowance such as a frowning face, and the method ends, as shown at step 35.

FIG. 3 is a flow diagram 40 of another exemplary embodiment of the present invention. In this example, the method is computer-implemented for use in a distributed computing environment, such as the Internet or World Wide Web. A user may select a restaurant where the user intends to dine, and receive a customized, user-specific, dietary restriction-specific, menu, e.g. via a graphical user interface. When a food item typically would not be prepared in a manner compatible with the dietary restriction, a preparation plan is prepared that makes the food item compatible with the dietary restriction. Accordingly, the user may determine whether a certain restaurant offers food items compatible with the user's dietary restriction. Advantageously, this may be determined before the user travels to the restaurant.

As shown in FIG. 3, the method starts with receipt of a signal identifying a dietary restriction, as shown at steps 41 and 42. For example, an individual may specify a dietary restriction and cause such a signal to be transmitted by selecting an objectionable ingredient (e.g. “milk”), an allergy (e.g. “dairy products”), or a classification (e.g. “kosher”) from a menu displayed on a video display screen of a distributed computing system.

The inventive system then references a database to identify preparation criteria associated with the dietary restriction, as shown as step 44 and as described above. For example, this database may be maintained by an administrator of the system, such as a restaurant manager. This preparation criteria will be checked against the preparation notes for food items on the menu to determine which, if any, food items are, or can be made, compatible with the dietary restriction.

The system then references a database to identify a first food item of the menu, and that food item's preparation notes are identified and compared, as shown at steps 46, 48 and 50 and as generally described above. For example, this database may be maintained by an administrator of the system, by a restaurant, by an independent party, etc. If the preparation notes and preparation criteria are compatible, the food item is approved for consumption in compliance with the dietary restriction, as shown at steps 52 and 54. The method then repeats for a next food item until all food items of the menu have been considered, as shown at step 62.

If it is determined that the preparation notes and preparation criteria are not compatible, then it is determined whether a preparation plan compatible with the preparation criteria can be prepared, if any, as shown at steps 52 and 56. If feasible, a compatible preparation plan is prepared and the food item is conditionally approved for consumption in compliance with the dietary restriction, the condition being preparation according to the food preparation plan, as shown at steps 58 and 60.

For the purpose of preparing a preparation plan, the preparation notes may indicate whether certain ingredients and/or characteristics are optional or may be excluded, or if certain ingredients cannot be excluded, etc. This data may be stored in a database maintained by the restaurant, system administrator, independent party, etc. For example, the preparation plan may indicate that tomato is an optional ingredient that can be excluded from a sandwich. Accordingly, the sandwich may be conditionally approved for consumption by an individual allergic to tomatoes and a preparation plan may be provided that instruction exclusion of the tomato when the sandwich is prepared for the individual. However, if the sandwich includes a hamburger patty that includes a mixture of beef and spices, and that hamburger patty is prepared to include those spices before it reaches the restaurant, the spices cannot be separated from the beef, and so the spices are not optional ingredients and cannot be excluded. Accordingly, the sandwich cannot be conditionally approved for consumption by an individual allergic to the spices because a preparation plan cannot be provided that separates the spices from the beef. It should be noted that the preparation plans may be as elaborate as desired, e.g. to offer a substitution of spice-free chicken for the hamburger patty, or to consider that the restaurant has beef available for other meals that could be used to make a spice-free hamburger patty. These and other alternatives will be apparent to those skilled in the art.

Steps 48-60 repeat for next food items until all of the food items of the menu have been considered, as shown at step 62.

When all food items have been considered, a customized menu is prepared, as shown at step 64. In this embodiment, the menu displays only the food items that are approved, and the food items that are conditionally approved. Preferably, each of the conditionally approved food items is shown with a corresponding preparation plan, which may be provided to the restaurant by the user. Alternatively, the preparation plan may be provided at the restaurant, e.g. by a display on a video display screen in the restaurant's kitchen to assist a chef in preparing the food item in compliance with the dietary restriction. Alternatively, the preparation plan may be displayed to a user on a video display screen of a home PC, a handheld device such as a personal digital assistant or Web-enable wireless telephone, or a restaurant's computer system, or may be printed on a menu or instruction sheet, etc. using a printer of a computer system. Optionally, the customized menu displays food items with indicia or highlighting indicating whether each food item of the menu is approved or disallowed. For example, the customized menu may be printed or displayed on a video display screen.

It should be noted that the method of FIG. 3 is also applicable to food items offered by multiple providers, e.g. multiple restaurants. The method is also applicable to multiple prepackaged food items, including food items offered by multiple suppliers, although it may be unlikely that preparation plans can be developed for prepackaged items. For example, the system may be used to consider all candy bars of various manufacturers to determine which ones are compatible with a dietary restriction.

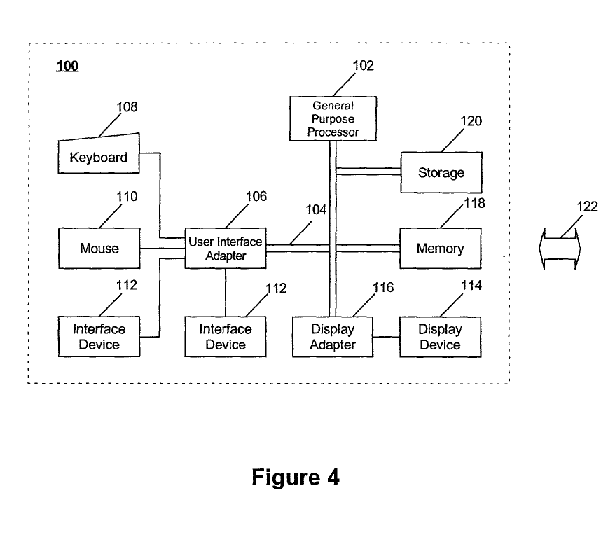

FIG. 4 is a block diagram of an information processing system in accordance with the present invention. As is well known in the art, the information processing system of FIG. 4 includes a general purpose microprocessor (CPU) 102 and a bus 104 employed to connect and enable communication between the microprocessor 102 and the components of the information processing system 100 in accordance with known techniques. The information processing system 100 typically includes a user interface adapter 106, which connects the microprocessor 102 via the bus 104 to one or more interface devices, such as a keyboard 108, mouse 110, and/or other interface devices 112, which can be any user interface device, such as a touch sensitive screen, digitized entry pad, etc. The bus 104 also connects a display device 114, such as an LCD screen or monitor, to the microprocessor 102 via a display adapter 116. The bus 104 also connects the microprocessor 102 to memory 118 and long-term storage 120 (collectively, “memory”) which can include a hard drive, diskette drive, tape drive, etc.

The information processing system 100 may communicate with other computers or networks of computers, for example via a communications channel, network card or modem 122. The information processing system 100 may be associated with such other computers in a local area network (LAN) or a wide area network (WAN), or the information processing system 100 can be a client or server in a client/server arrangement with another computer, etc. All of these configurations, as well as the appropriate communications hardware and software, are known in the art.

Software programming code for carrying out the inventive method is typically stored in memory. Accordingly, the information processing system 100 stores in its memory microprocessor executable instructions. These instructions may include a first program for identifying preparation criteria associated with the dietary restriction, a second program for identifying preparation notes for the food item, a third program stored for comparing the preparation criteria to the preparation notes, and a fourth program for providing a dietary plan for consumption of the food item that complies with the dietary restriction.

Additionally, computer readable media storing computer readable code for facilitating compliance with a dietary restriction is provided. The computer readable media stores code for carrying out first subprocesses for identifying preparation criteria associated with the dietary restriction, second subprocesses for identifying preparation notes for the food item, third subprocesses for comparing the preparation criteria to the preparation notes, and fourth subprocesses for providing a dietary plan for consumption of the food item that complies with the dietary restriction.

A computer program product recorded on a computer readable medium for facilitating compliance with a dietary restriction is also provided. The computer program product comprises computer readable means for identifying preparation criteria associated with the dietary restriction, computer readable means for identifying preparation notes for the food item, computer readable means for comparing the preparation criteria to the preparation notes, and computer readable means for providing a dietary plan for consumption of the food item that complies with the dietary restriction.

It will be appreciated that a graphical user interface may be advantageously provided to implement the present invention.

Having thus described particular embodiments of the invention, various alterations, modifications, and improvements will readily occur to those skilled in the art. Such alterations, modifications and improvements as are made obvious by this disclosure are intended to be part of this description though not expressly stated herein, and are intended to be within the spirit and scope of the invention. Accordingly, the foregoing description is by way of example only, and not limiting. The invention is limited only as defined in the following claims and equivalents thereto.

What is claimed is:

1. A method for facilitating compliance with a dietary restriction, the method comprising the steps of:
identifying preparation criteria associated with the dietary restriction;
identifying preparation notes for the food item;
comparing the preparation criteria to the preparation notes; and
providing a dietary plan for consumption of the food item that complies with the dietary restriction.

2. The method of claim 1, wherein the dietary plan indicates approval of consumption of the food item if the preparation criteria and preparation notes are compatible.

3. The method of claim 1, wherein the dietary plan indicates disallowance of consumption of the food item if the preparation criteria and preparation notes are not compatible.

4. The method of claim 1, further comprising the steps of:
determining that the preparation criteria and preparation notes are not compatible; and
preparing a preparation plan for the food item that is compatible with the preparation criteria;
wherein the dietary plan approves consumption of the food item when prepared according to the preparation plan.

5. The method of claim 1, wherein the providing step comprises:
preparing a menu identifying the food item.

6. The method of claim 5, wherein the providing step comprises:
displaying the menu on a video display screen.

7. The method of claim 1, wherein the providing step comprises:
displaying indicia on a video display screen.

8. The method of claim 1, further comprising the step of:
determining that the preparation criteria and preparation notes are not compatible;
wherein the providing step comprises the steps of:
referencing the preparation notes, the preparation notes identifying optional ingredients that may be excluded; and
preparing a preparation plan for the food item, the preparation plan comprising instructions to exclude at least one optional ingredient, the preparation plan making the food item compatible with the dietary restriction;
wherein the dietary plan indicates approval of consumption of the food item when prepared according to the preparation plan.

9. The method of claim 1, wherein the step of identifying the preparation criteria comprises referencing a database of related ingredients to identify a related ingredient associated with an ingredient associated with the dietary restriction.

10. A method for preparing a customized menu of food items that is compatible with a dietary restriction, the method comprising the step of:
identifying preparation criteria associated with the dietary restriction;
identifying preparation notes for each of a plurality of food items;
comparing the preparation criteria to the preparation notes for each of the plurality of food items; and
preparing a menu identifying any of said plurality of food items having respective preparation criteria that is compatible with respective preparation notes.

11. The method of claim 10, further comprising the steps of:
determining that the preparation criteria and preparation notes of a food item are not compatible; and
preparing a preparation plan for the food item that is compatible with the preparation criteria;
wherein the menu identifies the food item and the preparation plan.

12. The method of claim 10, wherein the step of identifying the preparation criteria comprises referencing a database of dietary restrictions to identify an ingredient associated with the dietary restriction;

13. The method of claim 10, wherein the step of identifying the preparation criteria comprises referencing a database of dietary restrictions to identify a preparation procedure associated with the dietary restriction.

14. The method of claim 12, wherein the step of identifying the preparation criteria comprises referencing a database of related ingredients to identify a related ingredient associated with the ingredient.

15. The method of claim 10, wherein the step of comparing comprises the steps of:
identifying an objectionable ingredient of the preparation notes for a food item;
determining whether the objectionable ingredient is optional; and
preparing a preparation plan to exclude the objectionable ingredient if the objectionable ingredient is optional; and
wherein the step of preparing the menu comprises displaying the preparation plan in association with the food item.

16. A method for facilitating compliance with a dietary restriction, the method comprising the steps of:
identifying preparation criteria associated with the dietary restriction;
identifying preparation notes for the food item;
comparing the preparation criteria to the preparation notes for a food item to determine whether they are compatible; and
if the preparation criteria and the preparation notes are compatible, approving the food item for consumption;
if the preparation criteria and the preparation notes are not compatible, preparing a preparation plan that makes the food item compatible with the preparation criteria, and
providing a dietary plan for consumption of the food item that is compatible with the dietary restriction.

17. A method for facilitating compliance with a dietary restriction using a computerized information processing system, the method comprising the steps of:
receiving a signal identifying a characteristic associated with the dietary restriction;
referencing a database to identify related characteristics associated with the characteristic;
identifying preparation notes for a food item, the preparation notes identifying characteristics of the food item;
comparing the preparation notes to the characteristic and any related characteristics; and
selectively approving the food item for consumption in compliance with the dietary restriction if the preparation notes are compatible with the characteristic and any related characteristics.

18. The method of claim 17, further comprising the steps of:
preparing a preparation plan that makes the food item compatible with the characteristic and any related characteristics if the preparation notes are not compatible with the characteristic and any related characteristics; and
conditionally approving the food item for consumption in compliance with the dietary restriction when the food item is prepared according to the preparation plan.

19. The method of claim 18, wherein the step of preparing comprises:
determining whether the characteristic and any related characteristics are optional for the food item; and
preparing a preparation plan comprising instructions to exclude the characteristic and any related characteristics if they are optional.

20. An information processing system for facilitating compliance with a dietary restriction, the system comprising:
a central processing unit (CPU) for executing programs;
a memory operatively connected to said CPU;
a first program stored in said memory and executable by said CPU for identifying preparation criteria associated with the dietary restriction;
a second program stored in said memory and executable by said CPU for identifying preparation notes for the food item;
a third program stored in said memory and executable by said CPU for comparing the preparation criteria to the preparation notes; and
a fourth program stored in said memory and executable by said CPU for providing a dietary plan for consumption of the food item that complies with the dietary restriction.

21. Computer readable media storing computer readable code for facilitating compliance with a dietary restriction, comprising:
first subprocesses for identifying preparation criteria associated with the dietary restriction;
second subprocesses for identifying preparation notes for the food item;
third subprocesses for comparing the preparation criteria to the preparation notes; and
fourth subprocesses for providing a dietary plan for consumption of the food item that complies with the dietary restriction.

22. A computer program product recorded on a computer readable medium for facilitating compliance with a dietary restriction comprising:
computer readable means for identifying preparation criteria associated with the dietary restriction;
computer readable means for identifying preparation notes for the food item;
computer readable means for comparing the preparation criteria to the preparation notes; and
computer readable means for providing a dietary plan for consumption of the food item that complies with the dietary restriction.
